(19) United States
(12) Patent Application Publication
(10) Pub. No.: US 2026/0130727 A1
(43) Pub. Date: May 14, 2026

(54) INSTRUMENT ATTACHMENT ASSEMBLIES

(71) Applicant: INTUITIVE SURGICAL OPERATIONS, INC., Sunnyvale, CA (US)

(72) Inventors: David Bailey, Portola Valley, CA (US); Thomas Roberts, Sunnyvale, CA (US)

(21) Appl. No.: 19/384,422

(22) Filed: Nov. 10, 2025

Related U.S. Application Data
(60) Provisional application No. 63/719,320, filed on Nov. 12, 2024.

(51) Int. Cl. A61B 34/30; A61B 34/00; A61B 34/20

(52) U.S. Cl. CPC A61B 34/30; A61B 34/20; A61B 34/71; A61B 2034/2051; A61B 2034/2061; A61B 2034/301

(57) ABSTRACT

A medical system includes an instrument and an instrument carriage. An instrument attachment assembly includes a post including an end surface and an indent spaced from the defining a proximal surface. A carriage attachment assembly includes a gear having a engagement helix member and a latching helix member. The end surface of the post engages the engagement helix member when force is applied to bring the instrument attachment assembly toward the instrument carriage to rotate the gear. A latch surface of the latching helix member engages the proximal surface of the post in a latched position.

### CROSS-REFERENCE TO RELATED APPLICATION

This application claims the priority benefit of U.S. Provisional Application No. 63/719,320, filed Nov. 12, 2024, which is hereby incorporated by reference herein in its entirety.

### FIELD

Disclosed embodiments relate to robotic surgical systems and, in particular, attachment assemblies for attaching instruments within robotic surgical systems.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. One such minimally invasive technique is to use a flexible and/or steerable elongate device, such as a flexible catheter, that can be inserted into anatomic passageways and navigated toward a region of interest within the patient anatomy.

### SUMMARY

The following presents a simplified summary of various examples described herein and is not intended to identify key or critical elements or to delineate the scope of the claims.

In accordance with a first example, a medical system is disclosed herein that includes an instrument carriage configured to control movement of an instrument. The instrument carriage includes a carriage attachment assembly configured to removably attach to the instrument. The carriage attachment assembly includes a gear including a body having an outer surface, an engagement helix member of the gear extending along a first portion of the outer surface of the body; and a latching helix member of the gear extending along a second portion of the outer surface of the body. The engagement helix member has an engagement surface configured to facilitate rotation of the gear from an open position where the instrument is unattached from the carriage attachment assembly towards a latched position where the instrument is attached to the carriage attachment assembly. The latching helix member has a latch surface configured to engage the instrument in the latched position.

In accordance with a second example, a medical system is disclosed herein that includes an instrument attachment assembly coupled to an instrument. The instrument attachment assembly includes a post including an end surface and an indent proximally spaced from end surface the defining a proximal surface. The medical system further includes an instrument carriage configured to control movement of the instrument. The instrument carriage includes a carriage attachment assembly configured to removably attach to the instrument via the instrument attachment assembly. The carriage attachment assembly includes a gear including a body having an outer surface, an engagement helix member of the gear extending along a first portion of the outer surface of the body, and a latching helix member of the gear extending along a second portion of the outer surface of the body. The engagement helix member has an engagement surface configured to be engaged by the end surface of the post when force is applied to bring the instrument attachment assembly toward the instrument carriage to facilitate rotation of the gear from an open position where the instrument is unattached from the carriage attachment assembly towards a latched position where the instrument attachment assembly is attached to the carriage attachment assembly. The latching helix member has a latch surface configured to engage the proximal surface of the post in the latched position.

In accordance with a third example, a medical system is disclosed herein that includes an instrument having an elongate medical device and an instrument attachment assembly coupled to the elongate medical device. The instrument attachment assembly is configured to removably attach to an instrument carriage for movement control of the elongate medical device. The instrument attachment assembly includes a post including an end surface configured to facilitate rotation of a gear of the instrument carriage from an open position where the post is unattached from the instrument carriage towards a latched position where the post is attached to the instrument carriage and an indent proximally spaced from the end surface defining a proximal surface, the proximal surface configured to engage the gear in the latched position.

In accordance with a fourth example, a method for attaching a carriage attachment assembly of an instrument carriage that controls movement of an instrument to the instrument is disclosed herein that includes driving rotation of a gear of the carriage attachment assembly from an open position where the instrument is released from the carriage attachment assembly to a latched position where the instrument is attached to the carriage attachment assembly by engaging an engagement surface of an engagement helix member of the carriage attachment assembly extending along a first portion of an outer surface of the gear with a latch member of the instrument and, in the latched position, engaging a latch surface of a latching helix member of the carriage attachment assembly extending along a second portion of the outer surface of the gear with the latch member to attach the instrument to the carriage attachment assembly of the instrument carriage.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a medical system according to some embodiments.

FIG. 2A is a simplified diagram of a medical instrument system according to some embodiments.

FIG. 2B is a simplified diagram of a medical instrument including a medical tool within an elongate device according to some embodiments.

FIGS. 3A and 3B are simplified diagrams of side views of a patient coordinate space including a medical instrument mounted on an insertion assembly according to some embodiments.

FIG. 4A is a perspective view of a medical system including an instrument and a carriage attachment assembly according to some embodiments.

FIG. 4B is a perspective view a gear for the carriage attachment assembly of FIG. 4A.

FIG. 4C is a top plan view of the gear of FIG. 4B.

FIG. 4D is a perspective view of a biasing mechanism and gear for the carriage attachment assembly of FIG. 4A.

FIG. 4E is a perspective view of a release mechanism and gear for the carriage attachment assembly of FIG. 4A.

FIG. 4F is a perspective view of the carriage attachment assembly of FIG. 4A including the biasing mechanism of FIG. 4D and the release mechanism of FIG. 4E.

FIG. 4G is a cross-sectional view of the carriage attachment assembly of FIG. 4A including a housing, the biasing mechanism of FIG. 4D, and the release mechanism of FIG. 4E.

FIG. 4H is a top sectional perspective view of the carriage attachment assembly of FIG. 4G.

FIG. 4I is a side cross-sectional view of a housing for an instrument attachment assembly of the medical system of FIG. 4A.

FIG. 4J is a side elevational view of the medical system of FIG. 4A.

FIG. 4K is a cross-sectional view of the medical system of FIG. 4A showing an instrument attachment assembly attached to a carriage attachment assembly.

FIG. 4L is a cross-sectional view of the medical system of FIG. 4A showing an instrument attachment assembly and a carriage attachment assembly in an open position.

FIG. 4M is a cross-sectional view of the medical system of FIG. 4A showing an instrument attachment assembly and a carriage attachment assembly engaging one another.

FIG. 4N is a cross-sectional view of the medical system of FIG. 4A showing an instrument attachment assembly and a carriage attachment assembly in a latched position.

FIG. 4O is a cross-sectional view of a second example medical system including an instrument attachment assembly and a carriage attachment assembly.

FIG. 5 is a flowchart illustrating a method for attaching and detaching an instrument to an instrument carriage according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

A medical system is provided with an attachment assembly that selectively couples an instrument, such as a flexible elongate device assembly, to an instrument carriage (e.g., of an instrument manipulator). In some examples, the instrument manipulator can provide for insertion and retraction of a flexible elongate device of the flexible elongate device assembly, with respect to patient anatomy, by moving the instrument carriage in a telescoping manner. The attachment assembly can be utilized to selectively couple the flexible elongate device assembly to the instrument carriage. The attachment assembly as described herein advantageously has larger tolerances for locating mating surfaces by utilizing more area for latch contact than some conventional assemblies. The attachment assembly is also self-locking and resists being back driven, ensuring a securing attachment that can resist user imparted loads during a procedure, as well as loads imparted by the instrument manipulator.

The attachment assembly can include a carriage attachment assembly having a gear (e.g., a worm gear) with engagement and latching helix members extending therearound. The engagement helix member can be utilized to facilitate rotation of the gear from an open, unlatched position towards a latched position. The carriage attachment assembly can also include, in some examples, a biasing mechanism to aid in facilitating rotation of the gear. The biasing mechanism can, at least partially (e.g., after an initial rotation range), facilitate rotation of the gear fully to the latched position where a latch surface of the latching helix member engages a surface coupled to the instrument.

In some examples, the carriage attachment assembly can include a release mechanism that facilitates rotation of the gear from the latched position towards the open position to thereby release the instrument from the carriage attachment assembly. Further, a biasing mechanism, such as that described above, can have a first range of gear rotation that facilitates rotation of the gear towards the latched position and a second range of gear rotation that facilitates rotation of the gear towards the open position. With this configuration, after the release mechanism facilitates rotation of the gear to a position within the second range of the biasing mechanism, the biasing mechanism facilitates rotation of the gear fully to the open position to release the instrument from the carriage attachment assembly.

In some examples, the attachment assembly can include an instrument attachment assembly with a post configured to engage the gear of the carriage attachment assembly. The post can include a distal surface that initially engages the engagement helix member to facilitate rotation of the gear when a force is asserted to bring the instrument towards the instrument carriage (e.g., inserting the post along the gear generally parallel to a rotational axis thereof). The post further includes an indent disposed above the distal surface that defines a proximal surface configured to engage a latch surface of the latching helix member as the gear is rotated to the latched position. During release, the gear is rotated to disengage the latching helix member from the proximal surface and drive the instrument attachment assembly away from the gear with the engagement helix member.

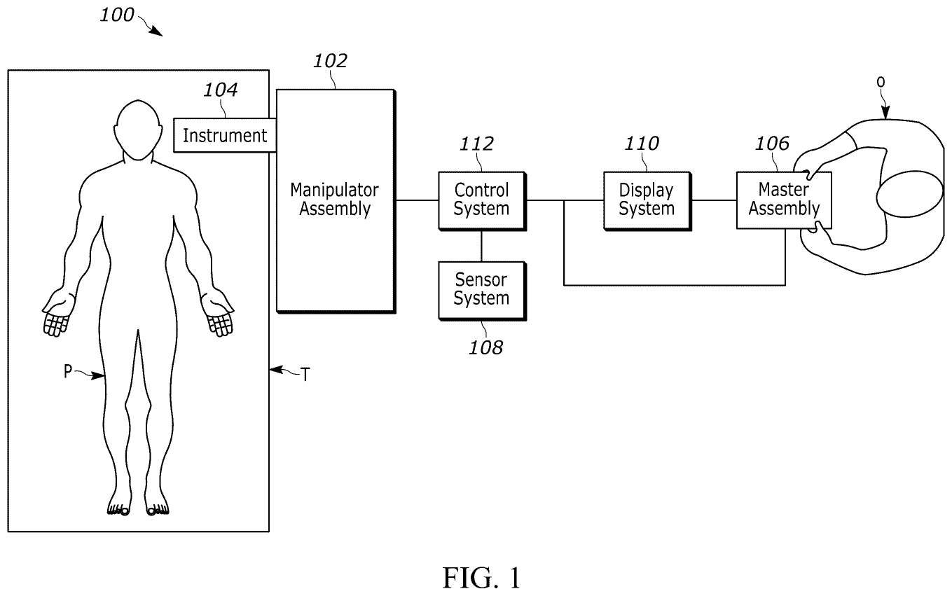

FIG. 1 is a simplified diagram of a medical system 100 according to some embodiments. The medical system 100 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 1, medical system 100 may include a manipulator assembly 102 that controls the operation of a medical instrument 104 in performing various procedures on a patient P. Medical instrument 104 may extend into an internal site within the body of patient P via an opening in the body of patient P. The manipulator assembly 102 may be teleoperated, non-teleoperated, or a hybrid teleoperated and non-teleoperated assembly with one or more degrees of freedom of motion that may be motorized and/or one or more degrees of freedom of motion that may be non-motorized (e.g., manually operated). The manipulator assembly 102 may be mounted to and/or positioned near a patient table T. A master assembly 106 allows an operator O (e.g., a surgeon, a clinician, a physician, or other user) to control the manipulator assembly 102. In some examples, the master assembly 106 allows the operator O to view the procedural site or other graphical or informational displays. In some examples, the manipulator assembly 102 may be excluded from the medical system 100 and the instrument 104 may be controlled directly by the operator O. In some examples, the manipulator assembly 102 may be manually controlled by the operator O. Direct operator control may include various handles and operator interfaces for hand-held operation of the instrument 104.

The master assembly 106 may be located at a surgeon's console which is in proximity to (e.g., in the same room as) a patient table T on which patient P is located, such as at the side of the patient table T. In some examples, the master assembly 106 is remote from the patient table T, such as in in a different room or a different building from the patient table T. The master assembly 106 may include one or more control devices for controlling the manipulator assembly 102. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 102 supports the medical instrument 104 and may include a kinematic structure of links that provide a set-up structure. The links may include one or more non-servo controlled links (e.g., one or more links that may be manually positioned and locked in place) and/or one or more servo controlled links (e.g., one or more links that may be controlled in response to commands, such as from a control system 112). The manipulator assembly 102 may include a plurality of actuators (e.g., motors) that drive inputs on the medical instrument 104 in response to commands, such as from the control system 112. The actuators may include drive systems that move the medical instrument 104 in various ways when coupled to the medical instrument 104. For example, one or more actuators may advance medical instrument 104 into a naturally or surgically created anatomic orifice. Actuators may control articulation of the medical instrument 104, such as by moving the distal end (or any other portion) of medical instrument 104 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 104, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 104.

The medical system 100 may include a sensor system 108 with one or more sub-systems for receiving information about the manipulator assembly 102 and/or the medical instrument 104. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 104; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 104 or from some other location; and/or actuator position sensors such as resolvers, encoders, potentiometers, and the like that describe the rotation and/or orientation of the actuators controlling the medical instrument 104.

The medical system 100 may include a display system 110 for displaying an image or representation of the procedural site and the medical instrument 104. Display system 110 and master assembly 106 may be oriented so physician O can control medical instrument 104 and master assembly 106 with the perception of telepresence.

In some embodiments, the medical instrument 104 may include a visualization system, which may include an image capture assembly that records a concurrent or real-time image of a procedural site and provides the image to the operator O through one or more displays of display system 110. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 104. Additionally or alternatively, a separate endoscope, attached to a separate manipulator assembly, may be used with medical instrument 104 to image the procedural site. The visualization system may be implemented as hardware, firmware, software or a combination thereof which interact with or are otherwise executed by one or more computer processors, such as of the control system 112.

Display system 110 may also display an image of the procedural site and medical instruments, which may be captured by the visualization system. In some examples, the medical system 100 provides a perception of telepresence to the operator O. For example, images captured by an imaging device at a distal portion of the medical instrument 104 may be presented by the display system 110 to provide the perception of being at the distal portion of the medical instrument 104 to the operator O. The input to the master assembly 106 provided by the operator O may move the distal portion of the medical instrument 104 in a manner that corresponds with the nature of the input (e.g., distal tip turns right when a trackball is rolled to the right) and results in corresponding change to the perspective of the images captured by the imaging device at the distal portion of the medical instrument 104. As such, the perception of telepresence for the operator O is maintained as the medical instrument 104 is moved using the master assembly 106. The operator O can manipulate the medical instrument 104 and hand controls of the master assembly 106 as if viewing the workspace in substantially true presence, simulating the experience of an operator that is physically manipulating the medical instrument 104 from within the patient anatomy.

In some examples, the display system 110 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 110 may display a virtual image that is generated based on tracking the location of medical instrument 104. For example, the tracked location of the medical instrument 104 may be registered (e.g., dynamically referenced) with the model generated using the pre-operative or intra-operative images, with different portions of the model correspond with different locations of the patient anatomy. As the medical instrument 104 moves through the patient anatomy, the registration is used to determine portions of the model corresponding with the location and/or perspective of the medical instrument 104 and virtual images are generated using the determined portions of the model. This may be done to present the operator O with virtual images of the internal procedural site from viewpoints of medical instrument 104 that correspond with the tracked locations of the medical instrument 104.

The medical system 100 may also include the control system 112, which may include processing circuitry that implements the some or all of the methods or functionality discussed herein. The control system 112 may include at least one memory and at least one processor for controlling the operations of the manipulator assembly 102, the medical instrument 104, the master assembly 106, the sensor system 108, and/or the display system 110. Control system 112 may include instructions (e.g., a non-transitory machine-readable medium storing the instructions) that when executed by the at least one processor, configures the one or more processors to implement some or all of the methods or functionality discussed herein. While the control system 112 is shown as a single block in FIG. 1, the control system 112 may include two or more separate data processing circuits with one portion of the processing being performed at the manipulator assembly 102, another portion of the processing being performed at the master assembly 106, and/or the like. In some examples, the control system 112 may include other types of processing circuitry, such as application-specific integrated circuits (ASICs) and/or field-programmable gate array (FPGAs). The control system 112 may be implemented using hardware, firmware, software, or a combination thereof.

In some examples, the control system 112 may receive feedback from the medical instrument 104, such as force and/or torque feedback. Responsive to the feedback, the control system 112 may transmit signals to the master assembly 106. In some examples, the control system 112 may transmit signals instructing one or more actuators of the manipulator assembly 102 to move the medical instrument 104. In some examples, the control system 112 may transmit informational displays regarding the feedback to the display system 110 for presentation or perform other types of actions based on the feedback.

The control system 112 may include a virtual visualization system to provide navigation assistance to operator O when controlling the medical instrument 104 during an image-guided medical procedure. Virtual navigation using the virtual visualization system may be based upon an acquired pre-operative or intra-operative dataset of anatomic passageways of the patient P. The control system 112 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 108 that is used to compute an (e.g., approximate) location of the medical instrument 104 with respect to the anatomy of patient P. The sensor system 108 may be used to register and display the medical instrument 104 together with the pre-operatively or intra-operatively recorded images. For example, PCT Publication WO 2016/191298 (published Dec. 1, 2016 and titled “Systems and Methods of Registration for Image Guided Surgery”), which is incorporated by reference herein in its entirety, discloses example systems.

During a virtual navigation procedure, the sensor system 108 may be used to compute the (e.g., approximate) location of the medical instrument 104 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 100 may further include operations and support systems (not shown) such as illumination systems, steering control systems, irrigation systems, and/or suction systems. In some embodiments, the medical system 100 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

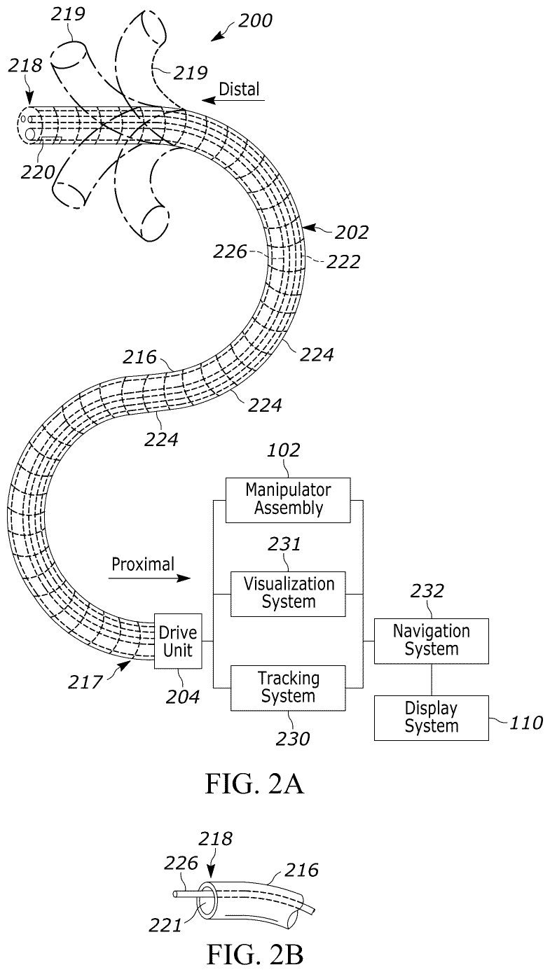

FIG. 2A is a simplified diagram of a medical instrument system 200 according to some embodiments. The medical instrument system 200 includes a flexible elongate device 202 (also referred to as elongate device 202), a drive unit 204, and a medical tool 226 that collectively is an example of a medical instrument 104 of a medical system 100. The medical system 100 may be a teleoperated system, a non-teleoperated system, or a hybrid teleoperated and non-teleoperated system, as explained with reference to FIG. 1. A visualization system 231, tracking system 230, and navigation system 232 are also shown in FIG. 2A and are example components of the control system 112 of the medical system 100. In some examples, the medical instrument system 200 may be used for non-teleoperational exploratory procedures or in procedures involving traditional manually operated medical instruments, such as endoscopy. The medical instrument system 200 may be used to gather (e.g., measure) a set of data points corresponding to locations within anatomic passageways of a patient, such as patient P.

The elongate device 202 is coupled to the drive unit 204. The elongate device 202 includes a channel 221 through which the medical tool 226 may be inserted. The elongate device 202 navigates within patient anatomy to deliver the medical tool 226 to a procedural site. The elongate device 202 includes a flexible body 216 having a proximal end 217 and a distal end 218. In some examples, the flexible body 216 may have an approximately 3 mm outer diameter. Other flexible body outer diameters may be larger or smaller.

Medical instrument system 200 may include the tracking system 230 for determining the position, orientation, speed, velocity, pose, and/or shape of the flexible body 216 at the distal end 218 and/or of one or more segments 224 along flexible body 216, as will be described in further detail below. The tracking system 230 may include one or more sensors and/or imaging devices. The flexible body 216, such as the length between the distal end 218 and the proximal end 217, may include multiple segments 224. The tracking system 230 may be implemented using hardware, firmware, software, or a combination thereof. In some examples, the tracking system 230 is part of control system 112 shown in FIG. 1.

Tracking system 230 may track the distal end 218 and/or one or more of the segments 224 of the flexible body 216 using a shape sensor 222. The shape sensor 222 may include an optical fiber aligned with the flexible body 216 (e.g., provided within an interior channel of the flexibly body 216 or mounted externally along the flexible body 216). In some examples, the optical fiber may have a diameter of approximately 200 μm. In other examples, the diameter may be larger or smaller. The optical fiber of the shape sensor 222 may form a fiber optic bend sensor for determining the shape of flexible body 216. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sep. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 216 may be determined using other techniques. For example, a history of the position and/or pose of the distal end 218 of the flexible body 216 can be used to reconstruct the shape of flexible body 216 over an interval of time (e.g., as the flexible body 216 is advanced or retracted within a patient anatomy). In some examples, the tracking system 230 may alternatively and/or additionally track the distal end 218 of the flexible body 216 using a position sensor system 220. Position sensor system 220 may be a component of an EM sensor system with the position sensor system 220 including one or more position sensors. Although the position sensor system 220 is shown as being near the distal end 218 of the flexible body 216 to track the distal end 218, the number and location of the position sensors of the position sensor system 220 may vary to track different regions along the flexible body 216. In one example, the position sensors include conductive coils that may be subjected to an externally generated electromagnetic field. Each coil of position sensor system 220 may produce an induced electrical signal having characteristics that depend on the position and orientation of the coil relative to the externally generated electromagnetic field. The position sensor system 220 may measure one or more position coordinates and/or one or more orientation angles associated with one or more portions of flexible body 216. In some examples, the position sensor system 220 may be configured and positioned to measure six degrees of freedom, e.g., three position coordinates X, Y, Z and three orientation angles indicating pitch, yaw, and roll of a base point. In some examples, the position sensor system 220 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999 and titled “Six-Degree of Freedom Tracking System Having a Passive Transponder on the Object Being Tracked”), which is incorporated by reference herein in its entirety.

In some embodiments, the tracking system 230 may alternately and/or additionally rely on a collection of pose, position, and/or orientation data stored for a point of an elongate device 202 and/or medical tool 226 captured during one or more cycles of alternating motion, such as breathing. This stored data may be used to develop shape information about the flexible body 216. In some examples, a series of position sensors (not shown), such as EM sensors like the sensors in position sensor 220 or some other type of position sensors may be positioned along the flexible body 216 and used for shape sensing. In some examples, a history of data from one or more of these position sensors taken during a procedure may be used to represent the shape of elongate device 202, particularly if an anatomic passageway is generally static.

FIG. 2B is a simplified diagram of the medical tool 226 within the elongate device 202 according to some embodiments. The flexible body 216 of the elongate device 202 may include the channel 221 sized and shaped to receive the medical tool 226. In some embodiments, the medical tool 226 may be used for procedures such as diagnostics, imaging, surgery, biopsy, ablation, illumination, irrigation, suction, electroporation, etc. Medical tool 226 can be deployed through channel 221 of flexible body 216 and operated at a procedural site within the anatomy. Medical instrument 226 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 226 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 226 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 221 when the biopsy tool is within the channel 221. The medical tool 226 may be an image capture probe that includes a distal portion with a stereoscopic or monoscopic camera that may be placed at or near the distal end 218 of flexible body 216 for capturing images (e.g., still or video images). The captured images may be processed by the visualization system 231 for display and/or provided to the tracking system 230 to support tracking of the distal end 218 of the flexible body 216 and/or one or more of the segments 224 of the flexible body 216. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 231. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 216 of the elongate device 202 to facilitate visual navigation of the elongate device 202 to a procedural site and then is replaced within the flexible body 216 with another type of medical tool 226 that performs the procedure. In some examples, the image capture probe may be within the flexible body 216 of the elongate device 202 along with another type of medical tool 226 to facilitate simultaneous image capture and tissue intervention, such as within the same channel 221 or in separate channels. A medical tool 226 may be advanced from the opening of the channel 221 to perform the procedure (or some other functionality) and then retracted back into the channel 221 when the procedure is complete. The medical tool 226 may be removed from the proximal end 217 of the flexible body 216 or from another optional instrument port (not shown) along flexible body 216.

In some examples, the elongate device 202 may include integrated imaging capability rather than utilize a removable image capture probe. For example, the imaging device (or fiber-optic bundle) and the light emitters may be located at the distal end 218 of the elongate device 202. The flexible body 216 may include one or more dedicated channels that carry the cable(s) and/or optical fiber(s) between the distal end 218 and the visualization system 231. Here, the medical instrument system 200 can perform simultaneous imaging and tool operations.

In some examples, the medical tool 226 is capable of controllable articulation. The medical tool 226 may house cables (which may also be referred to as pull wires), linkages, or other actuation controls (not shown) that extend between its proximal and distal ends to controllably bend the distal end of medical tool 226, such as discussed herein for the flexible elongate device 202. The medical tool 226 may be coupled to a drive unit 204 and the manipulator assembly 102. In these examples, the elongate device 202 may be excluded from the medical instrument system 200 or may be a flexible device that does not have controllable articulation. Steerable instruments or tools, applicable in some embodiments, are further described in detail in U.S. Pat. No. 7,316,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sep. 30, 2008 and titled “Passive Preload and Capstan Drive for Surgical Instruments”), which are incorporated by reference herein in their entireties.

The flexible body 216 of the elongate device 202 may also or alternatively house cables, linkages, or other steering controls (not shown) that extend between the drive unit 204 and the distal end 218 to controllably bend the distal end 218 as shown, for example, by broken dashed line depictions 219 of the distal end 218 in FIG. 2A. In some examples, at least four cables are used to provide independent up-down steering to control a pitch of the distal end 218 and left-right steering to control a yaw of the distal end 281. In these examples, the flexible elongate device 202 may be a steerable catheter. Examples of steerable catheters, applicable in some embodiments, are described in detail in PCT Publication WO 2019/018736 (published Jan. 24, 2019 and titled “Flexible Elongate Device Systems and Methods”), which is incorporated by reference herein in its entirety.

In embodiments where the elongate device 202 and/or medical tool 226 are actuated by a teleoperational assembly (e.g., the manipulator assembly 102), the drive unit 204 may include drive inputs that removably couple to and receive power from drive elements, such as actuators, of the teleoperational assembly. In some examples, the elongate device 202 and/or medical tool 226 may include gripping features, manual actuators, or other components for manually controlling the motion of the elongate device 202 and/or medical tool 226. The elongate device 202 may be steerable or, alternatively, the elongate device 202 may be non-steerable with no integrated mechanism for operator control of the bending of distal end 218. In some examples, one or more channels 221 (which may also be referred to as lumens), through which medical tools 226 can be deployed and used at a target anatomical location, may be defined by the interior walls of the flexible body 216 of the elongate device 202.

In some examples, the medical instrument system 200 (e.g., the elongate device 202 or medical tool 226) may include a flexible bronchial instrument, such as a bronchoscope or bronchial catheter, for use in examination, diagnosis, biopsy, and/or treatment of a lung. The medical instrument system 200 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 230 may be sent to the navigation system 232, where the information may be combined with information from the visualization system 231 and/or pre-operatively obtained models to provide the physician, clinician, surgeon, or other operator with real-time position information. In some examples, the real-time position information may be displayed on the display system 110 for use in the control of the medical instrument system 200. In some examples, the navigation system 232 may utilize the position information as feedback for positioning medical instrument system 200. Various systems for using fiber optic sensors to register and display a surgical instrument with surgical images, applicable in some embodiments, are provided in U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety.

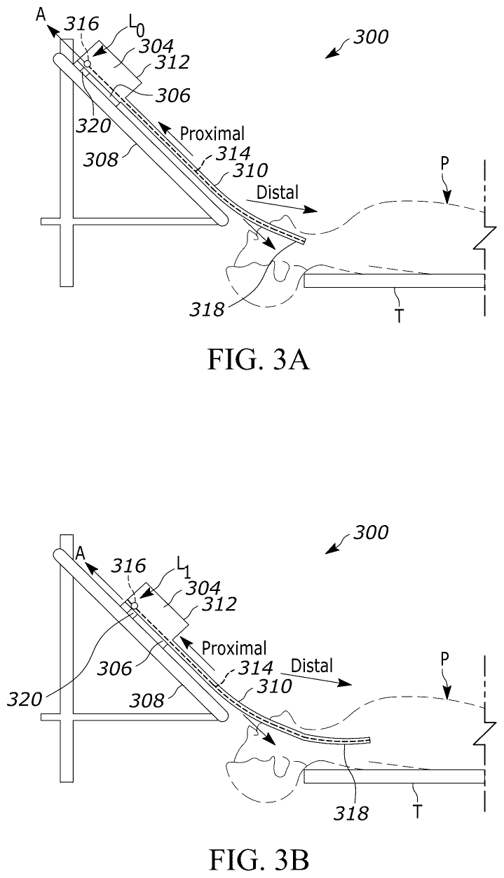

FIGS. 3A and 3B are simplified diagrams of side views of a patient coordinate space including a medical instrument mounted on an insertion assembly according to some embodiments. As shown in FIGS. 3A and 3B, a surgical environment 300 may include a patient P positioned on the patient table T. Patient P may be stationary within the surgical environment 300 in the sense that gross patient movement is limited by sedation, restraint, and/or other means. Cyclic anatomic motion, including respiration and cardiac motion, of patient P may continue. Within surgical environment 300, a medical instrument 304 is used to perform a medical procedure which may include, for example, surgery, biopsy, ablation, illumination, irrigation, suction, or electroporation. The medical instrument 304 may also be used to perform other types of procedures, such as a registration procedure to associate the position, orientation, and/or pose data captured by the sensor system 108 to a desired (e.g., anatomical or system) reference frame. The medical instrument 304 may be, for example, the medical instrument 104. In some examples, the medical instrument 304 may include an elongate device 310 (e.g., a catheter) coupled to an instrument body 312. Elongate device 310 includes one or more channels sized and shaped to receive a medical tool.

Elongate device 310 may also include one or more sensors (e.g., components of the sensor system 108). In some examples, a shape sensor 314 may be fixed at a proximal point 316 on the instrument body 312. The proximal point 316 of the shape sensor 314 may be movable with the instrument body 312, and the location of the proximal point 316 with respect to a desired reference frame may be known (e.g., via a tracking sensor or other tracking device). The shape sensor 314 may measure a shape from the proximal point 316 to another point, such as a distal end 318 of the elongate device 310. The shape sensor 314 may be aligned with the elongate device 310 (e.g., provided within an interior channel or mounted externally). In some examples, the shape sensor 314 may optical fibers used to generate shape information for the elongate device 310.

In some examples, position sensors (e.g., EM sensors) may be incorporated into the medical instrument 304. A series of position sensors may be positioned along the flexible elongate device 310 and used for shape sensing. Position sensors may be used alternatively to the shape sensor 314 or with the shape sensor 314, such as to improve the accuracy of shape sensing or to verify shape information.

Elongate device 310 may house cables, linkages, or other steering controls that extend between the instrument body 312 and the distal end 318 to controllably bend the distal end 318. In some examples, at least four cables are used to provide independent up-down steering to control a pitch of distal end 318 and left-right steering to control a yaw of distal end 318. The instrument body 312 may include drive inputs that removably couple to and receive power from drive elements, such as actuators, of a manipulator assembly.

The instrument body 312 may be coupled to an instrument carriage 306. The instrument carriage 306 may be mounted to an insertion stage 308 that is fixed within the surgical environment 300. Alternatively, the insertion stage 308 may be movable but have a known location (e.g., via a tracking sensor or other tracking device) within surgical environment 300. Instrument carriage 306 may be a component of a manipulator assembly (e.g., manipulator assembly 102) that couples to the medical instrument 304 to control insertion motion (e.g., motion along an insertion axis A) and/or motion of the distal end 318 of the elongate device 310 in multiple directions, such as yaw, pitch, and/or roll. The instrument carriage 306 or insertion stage 308 may include actuators, such as servomotors, that control motion of instrument carriage 306 along the insertion stage 308.

A sensor device 320, which may be a component of the sensor system 108, may provide information about the position of the instrument body 312 as it moves relative to the insertion stage 308 along the insertion axis A. The sensor device 320 may include one or more resolvers, encoders, potentiometers, and/or other sensors that measure the rotation and/or orientation of the actuators controlling the motion of the instrument carriage 306, thus indicating the motion of the instrument body 312. In some embodiments, the insertion stage 308 has a linear track as shown in FIGS. 3A and 3B. In some embodiments, the insertion stage 308 may have curved track or have a combination of curved and linear track sections.

FIG. 3A shows the instrument body 312 and the instrument carriage 306 in a retracted position along the insertion stage 308. In this retracted position, the proximal point 316 is at a position LO on the insertion axis A. The location of the proximal point 316 may be set to a zero value and/or other reference value to provide a base reference (e.g., corresponding to the origin of a desired reference frame) to describe the position of the instrument carriage 306 along the insertion stage 308. In the retracted position, the distal end 318 of the elongate device 310 may be positioned just inside an entry orifice of patient P. Also in the retracted position, the data captured by the sensor device 320 may be set to a zero value and/or other reference value (e.g., I=0). In FIG. 3B, the instrument body 312 and the instrument carriage 306 have advanced along the linear track of insertion stage 308, and the distal end 318 of the elongate device 310 has advanced into patient P. In this advanced position, the proximal point 316 is at a position L1 on the insertion axis A. In some examples, the rotation and/or orientation of the actuators measured by the sensor device 320 indicating movement of the instrument carriage 306 along the insertion stage 308 and/or one or more position sensors associated with instrument carriage 306 and/or the insertion stage 308 may be used to determine the position L1 of the proximal point 316 relative to the position LO. In some examples, the position L1 may further be used as an indicator of the distance or insertion depth to which the distal end 318 of the elongate device 310 is inserted into the passageway(s) of the anatomy of patient P.

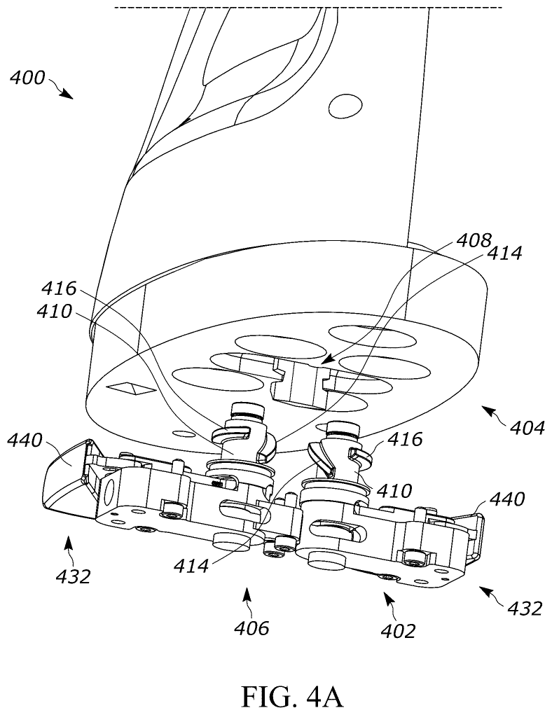

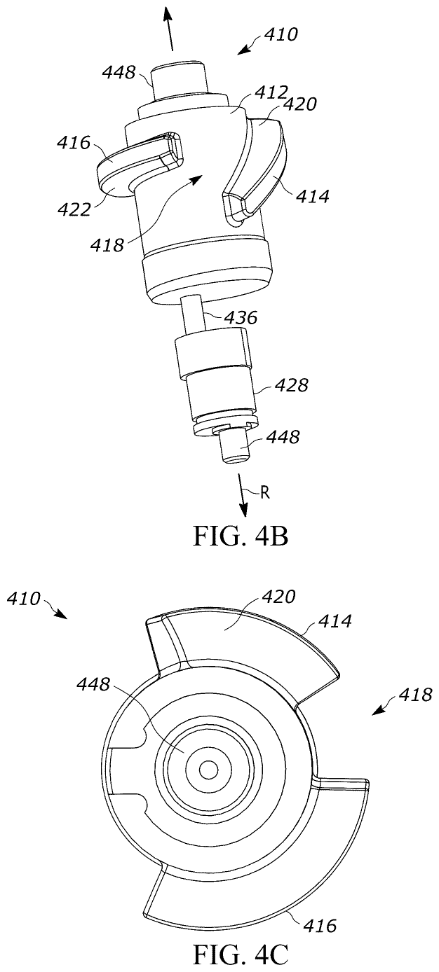

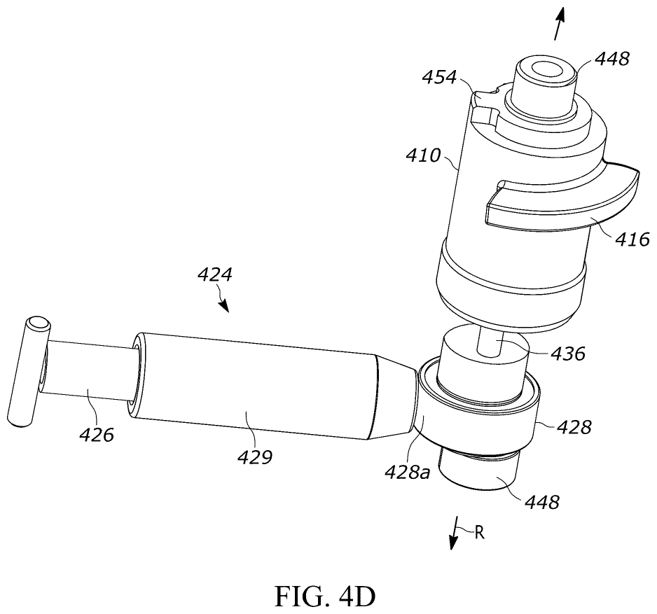

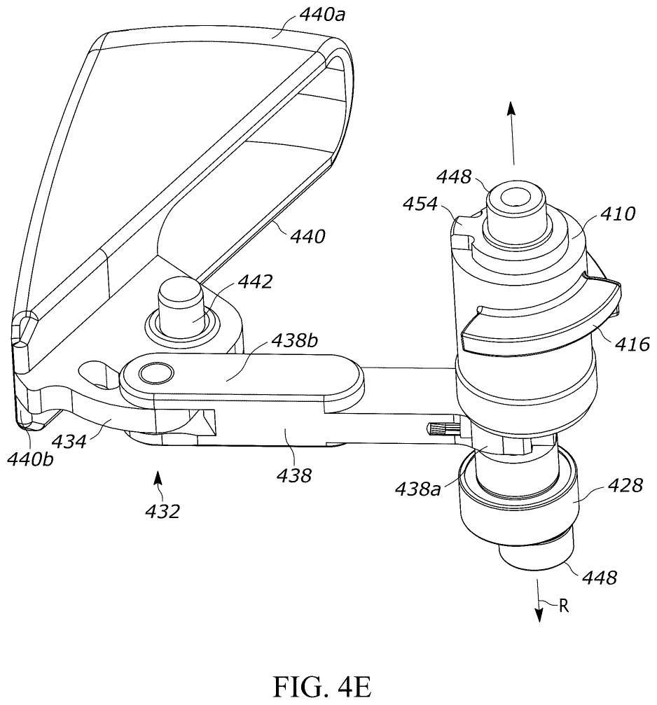

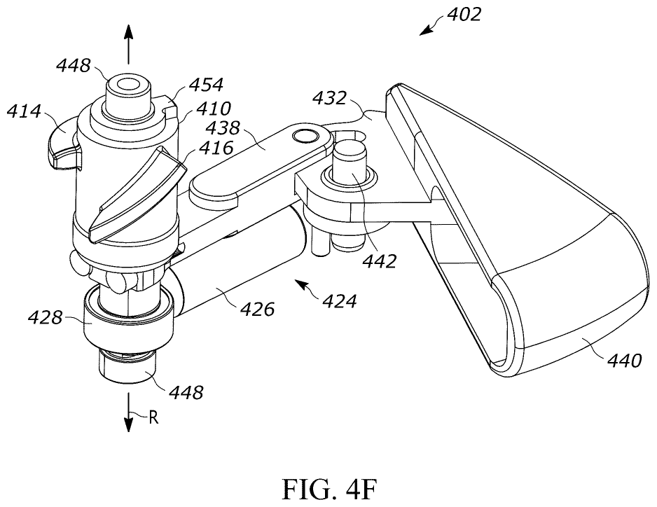

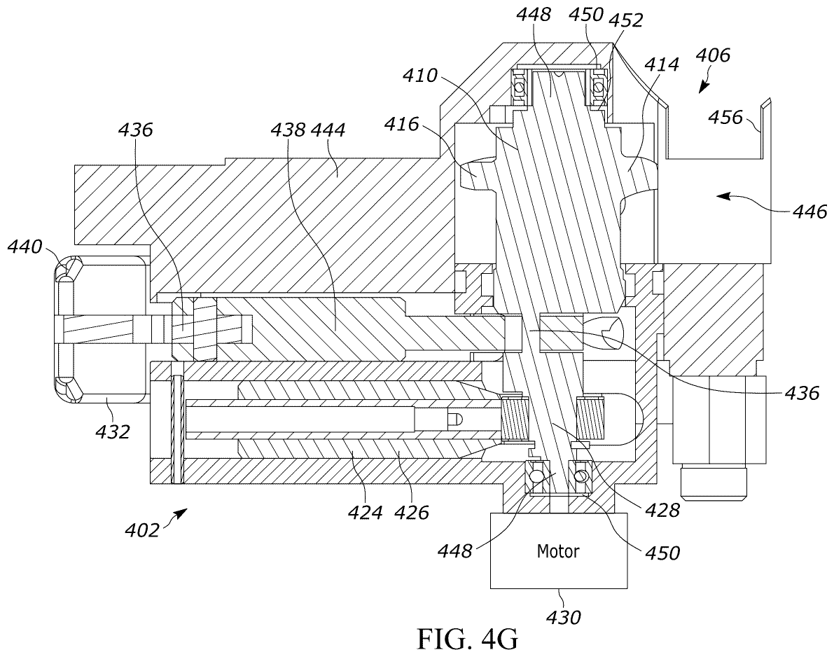

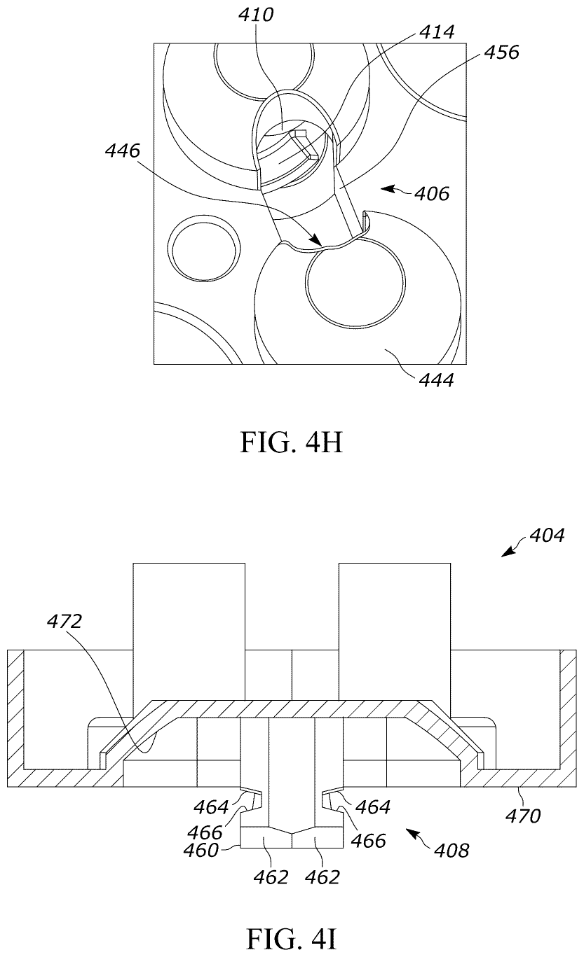

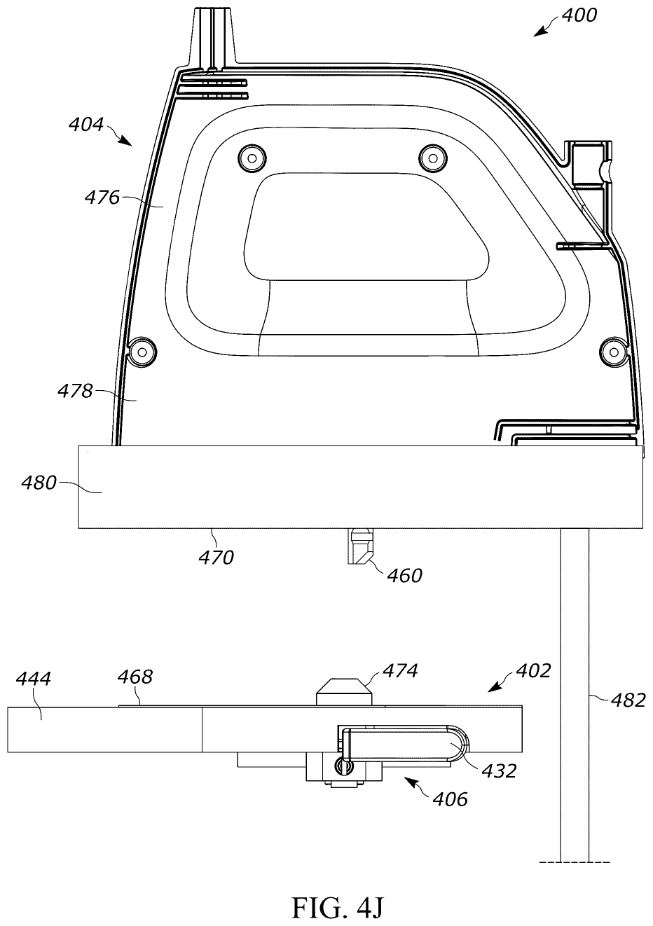

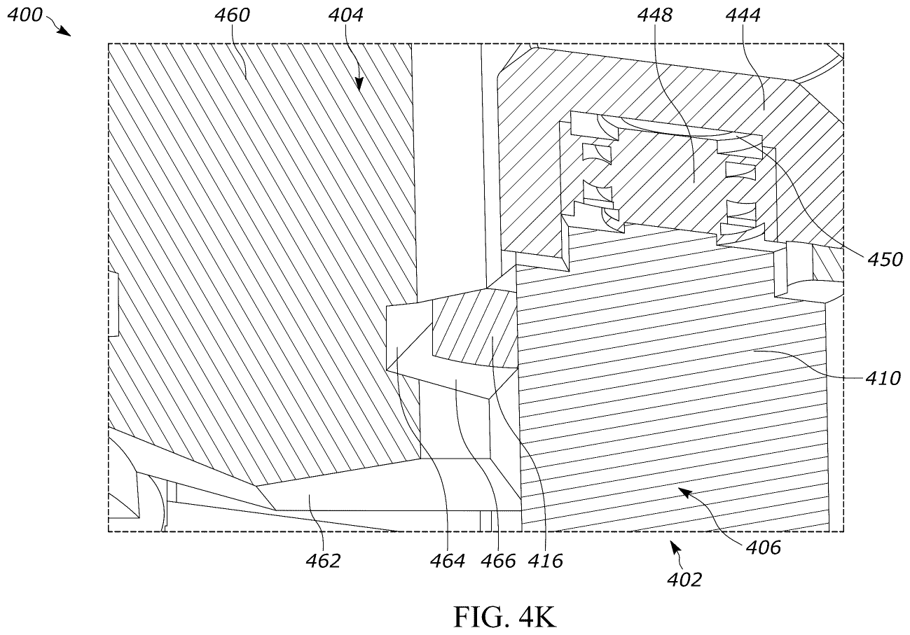

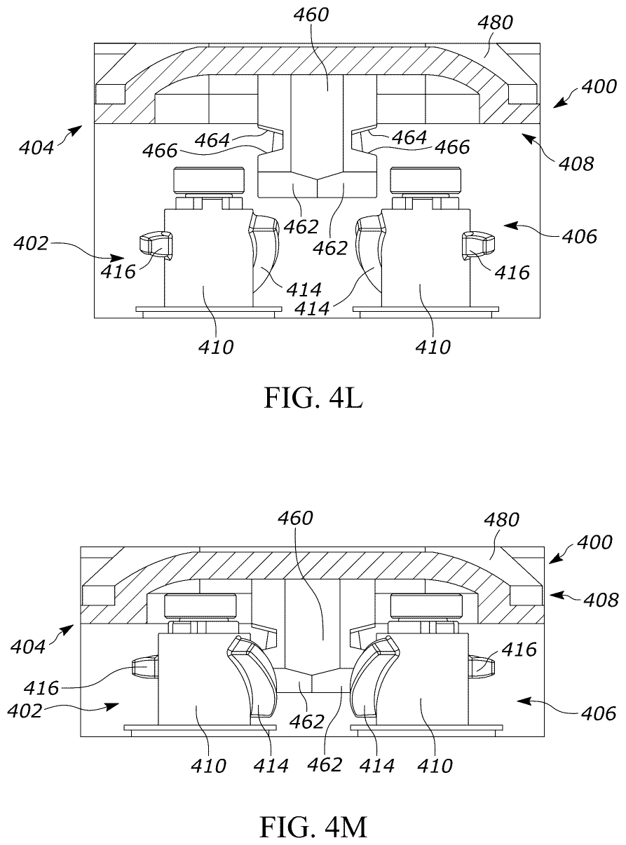

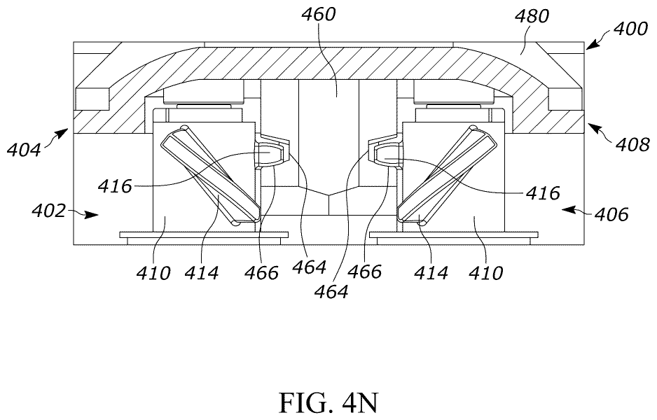

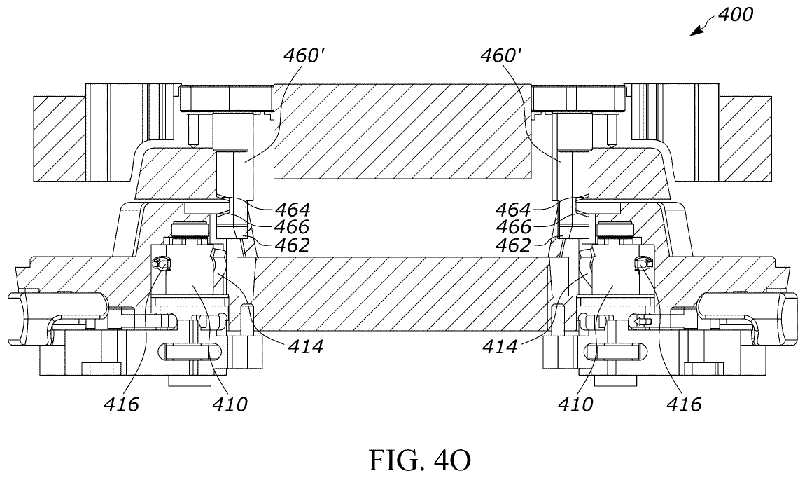

A medical system 400 is shown in FIGS. 4A-4N. According to some embodiments consistent with FIGS. 1-3, the medical system 400 may correspond to the medical instrument system 100, 200.

The medical system 400 includes an instrument carriage 402 and an instrument 404 that releasably couple together via a carriage attachment assembly 406 of the instrument carriage 402 and an instrument attachment assembly 408 of the instrument 404. The instrument carriage 402 is configured to control movement of the instrument 404 (e.g., distally and proximally) relative to a patient. Further, the coupling between the instrument carriage 402 and the instrument 404 can also connect one or more components of the system, such as sensor lines, articulation controls, etc.

As shown in FIGS. 4B and 4C, the carriage attachment assembly 406 includes a gear 410. The medical system 400 can include one gear 410 to attach the instrument carriage 402 to the instrument 404, two gears 410, or more. Each gear 410 is rotatable between an open position where the instrument 404 is unattached to the carriage attachment assembly 406 and a latched position where the instrument 404 is attached to the carriage attachment assembly 406 to mount the instrument 404 to the instrument carriage 402. In particular, the gear 410 of the carriage attachment assembly 406 in the latched position attaches to the instrument attachment assembly 408 of the instrument 404.

Each gear 410 includes a body having an outer surface 412, an engagement helix member 414, and a latching helix member 416. The engagement helix member 414 extends along a first portion of the outer surface 412 at a first ramp angle and the latching helix member 416 extends along a second portion of the outer surface 412 at a second ramp angle. In some examples, the first portion and the second portion of the outer surface 412 do not overlap such that the engagement helix member 414 and the latching helix member 416 extend over different circumferential areas of the gear 410 (e.g., do not longitudinally overlap). In further examples, the first portion and second portion of the outer surface 412 can be circumferentially spaced from one another along the outer surface 412 by a gap 418.

The engagement helix member 414 has an engagement surface 420 that can be engaged by structure of the instrument 404 (as discussed in greater detail in connection with FIG. 4I) to initiate or facilitate rotation of the gear 410 from the open position when mounting the instrument 404 to the instrument carriage 402. As shown, the angled engagement surface 420 faces upwardly (e.g., is accessible from above), at least partially oriented towards the instrument 404 as the instrument 404 is being mounted to the instrument carriage 402. As the gear 410 continues to rotate, a latch surface 422 of the latching helix member 416 engages structure of the instrument 404 to mount the instrument 404 to the instrument carriage 402. As shown, the latch surface 422 faces downwardly, at least partially away from the instrument 404 when first inserted, such that the latch surface 422 pulls the instrument 404 to the instrument carriage 402 as the gear 410 is rotated fully to the latched position. Thereafter, the latching helix member 416 can be disengaged from the structure of the instrument 404 by rotating the gear 410 in the opposite direction and, as the gear 410 rotates, the engagement helix member 414 can be used to eject or push the instrument 404 away from the instrument carriage 402.

In some examples, the ramp angle of the engagement helix member 414 is larger (e.g., steeper) than the ramp angle of the latching helix member 416. The relatively larger ramp angle of the engagement helix member 414 enables the instrument 404 to easily facilitate rotation of the gear 410 and slide therealong when being mounted to the instrument carriage 402. The relatively smaller ramp angle of the latching helix member 416 provides a latch with structure of the instrument 404 that has increased friction and stability. For example, the ramp angle of the engagement helix member 414 can be in a range of about 30 degrees to about 50 degrees, or about 39 or 40 degrees, and the ramp angle of the latching helix member 416 can be in a range of about 1 degree to about 10 degrees, or about 4 or 5 degrees. In some examples, the ramp angle of the latching helix member 416 can be selected as a function of the friction between the latch surface 422 and the proximal surface 466, where the friction is sufficient to prevent backdriving of the gear 410 when the instrument 404 is pulled.

As shown in FIG. 4D, the carriage attachment assembly 406 can include a biasing mechanism 424 that is operable to at least partially facilitate or drive rotation of the gear 410. For example, the biasing mechanism 424 is configured to at least partially facilitate or drive the gear 410 in a first direction to the latched position. In a further example, the biasing mechanism 424 is also configured to at least partially facilitate or drive the gear 410 in a second direction, opposite to the first direction, to the open position.

In one example, the biasing mechanism 424 is a spring mechanism having two stable end positions (corresponding with the open and latched positions) and a neutral (unstable) middle position. The biasing mechanism 424 includes a bias member 426, such as a compression spring, and so forth, that is coupled to a first journal 428 of the gear 410. A housing 429 can be disposed over the bias member 426 to provide a drive member for the bias member 426 and a surface to engage the first journal 428. The bias member 426 may also include a sleeve extending therearound to be disposed between the bias member 426 and the housing 429. The housing 429 and sleeve may be made from a low friction material, such as plastic, for ease of motion (e.g., relative rotation of the journal 428). The first journal 428 has a configuration that is eccentric to (e.g., having a center offset from) the rotation axis R. The bias member 426 engages the first journal to apply a biasing force to the first journal 428 that facilitates or drives rotation of the gear 410.

As shown in FIG. 4D, the eccentric configuration of the first journal 428 results in a larger offset portion 428a of the journal 428 relative to the rotation axis R of the gear 410. When the circumferential location of the journal 428 corresponding to a largest radius about the rotation axis R of the journal 428 is aligned with the bias member 426, the biasing mechanism 424 is in the middle, neutral position and the bias member 426 is held in a maximally compressed state, but does not urge rotation of the gear 410 in either direction. This is because the bias member 426 applies a biasing force that is parallel to its longitudinal axis and in this configuration, the biasing force is directed through the rotational axis R of the gear 410. When the gear 410 is rotated from this position, however, the direction of the biasing force from the compressed bias member 426 is offset from the longitudinal/rotational axis R of the gear 410 and the biasing force urges the gear 410 to rotate in the direction of the corresponding to the offset of the biasing force (i.e., toward the open position or the latched position).

With the above configuration, the biasing mechanism 424 has a first range of gear rotation that urges rotation of the gear 410 to the open position (and stably holds the gear 410 in the open position) and a second range of gear rotation that urges rotation of the gear 410 to the latched position (and stably holds the gear 410 in the latched position). The first and second ranges are disposed on either side of and separated by the neutral position. Accordingly, when first mounting the instrument 404 to the instrument carriage 402, the instrument 404 can be used to rotate the gear 410 to and past the neutral position of the biasing mechanism 424. After the gear 410 is rotated past the neutral position of the biasing mechanism 424, the biasing mechanism 424 urges rotation of the gear 410 to the latched position. As such, during latching, the gear 410 needs to be driven against the biasing force of the biasing mechanism 424 within the first range and the biasing mechanism 424 aids in rotation of the gear 410 to the latched position in the second range. Similarly, during release, the gear 410 is rotated against the force of the biasing mechanism 424 within the second range and the biasing mechanism 424 aids in rotation of the gear 410 to the open position in the first range.

In examples with or without the biasing mechanism 424, the medical system 400 can include a motor 430 that is operably coupled to and configured to drive rotation of the gear 410 between the open and latched positions according to signals from a control system of the medical system 400.

As shown in FIG. 4E, the carriage attachment assembly 406 includes a release mechanism 432 that is coupled to the gear 410 and configured to drive rotation of the gear 410 from the latched position towards the open position.

The release mechanism 432 includes a linkage 434 that is coupled to a second journal 436 (shown in FIG. 4D) of the gear 410. The second journal 436 has a configuration that is eccentric to (e.g., having a center offset from) the rotation axis R of the gear 410. The linkage 434 is configured to be manipulated by a user to apply a force to the second journal 436 that is offset from the rotational axis R of the gear 410 on a side that facilitates or drives rotation of the gear 410 from the latched position toward the open position.

In some examples, the release mechanism 432 is provided in the medical system 400 in addition to the biasing mechanism 424. In these examples, a user manipulates the release mechanism 432 to rotate the gear 410 against the force of the biasing mechanism 424 in the second range past the neutral position. Thereafter, the biasing mechanism 424 facilitates or drives rotation of the gear 410 to the open position.

As shown in FIG. 4E, the linkage 434 of the release mechanism 432 includes an arm 438 and a lever 440. The arm 438 has a first end 438a coupled to (e.g., encircling, fixed around, etc.) the second journal 436 and a second end 438b pivotably coupled to the lever 440. The lever 440 is pivotable about a pivot connection 442 and includes a distal, manipulation end 440a and a proximal end 440b on opposite sides of the pivot connection 442. The arm 438 is pivotably coupled to the proximal end 440b of the lever 440, such that rotation of the manipulation end 440a in a direction generally towards the gear 410 shifts the arm 438 in a direction generally away from the gear 410 and shifting of the arm 438 towards the gear 410 rotates the manipulation end 440a in a direction generally away from the gear 410.

With this configuration, as the gear 410 is rotated to the latched position, the arm 438 is drawn toward the gear 410 due to the eccentric configuration of the second journal 436, which causes the lever 440 to pivot about the pivot connection 442 and the manipulation end 440a to move outwardly. Thereafter, when release of the instrument 404 is desired, a user can push on the manipulation end 440a of the lever 440 to cause the lever 440 to pivot about the pivot connection 442 and draw the arm 438 outwardly, away from the gear 410. The eccentric configuration of the second journal 436 drives or urges the gear 410 to rotate from the latched position towards the open position. The release mechanism 432 (e.g., the configuration of the second journal 436, pivot range of the lever 440, and so forth) can be configured to drive the gear 410 fully from the latched position to past the neutral position of the biasing mechanism 424. This configuration advantageously prevents the carriage attachment assembly 406 from being driven back to a latched configuration as a result of motion of the release mechanism 432.

As shown in FIGS. 4G and 4H, the instrument carriage 402 includes a housing 444 for the carriage attachment assembly 406. The housing 444 defines an interior 446 sized to rotatably receive the gear 410 therein. In some examples, to rotatably mount the gear 410 in the housing 444 and hold the gear 410 in a desired (e.g., an upright) orientation, the gear 410 includes top and bottom mounts 448 that are rotatably received within cavities 450 defined by the housing 444. For example, the top and bottom mounts 448 can be cylindrical pins and the cavities 450 can have a complementary cylindrical shape. The housing 444 can include a number of housing portions that secure together by any suitable mechanism (e.g., fasteners, adhesive, snap-fit connections, tongue-and-groove connections, welding, and so forth) to mount components of the instrument carriage 402 therein.

The housing 444 optionally has a configuration that constrains rotation of the gear 410 within the housing interior 446. For example, as shown, the housing 444 defines a first stop surface 452 that structure 454 of the gear 410 abuts when the gear 410 rotates to and is in the open position. The structure 454 can be a separate protrusion as shown in FIG. 4D or can be other suitable structure, such as the engagement helix member 414, the latching helix member 416, etc. With this configuration, the biasing mechanism 424 is sized and configured to bias the gear 410 to hold the structure 454 against the first stop surface 452 to effectively hold the gear 410 in the open position. In other examples, the first stop surface 452 can be configured to engage structure of the biasing mechanism 424, the release mechanism 432, or other component coupled to the gear 410.

The housing 444 further defines an access opening 456 aligned over the gear 410 to expose the engagement surface 420 of the engagement helix member 414 when the gear 410 is in the open position and allow engagement of the gear 410 therethrough. With this configuration, structure of the instrument 404, discussed in more detail below, can be inserted through the access opening 456 to sequentially engage the engagement helix member 414 followed by the latching helix member 416 to secure the instrument attachment assembly 408 to the carriage attachment assembly 406.

In some examples, more than one gear 410 may be desired for additional latch points. Accordingly, the medical system 400 can utilize one gear 410, two gears 410, three gears 410, four gears 410, or more. Adjacent gears 410 and release mechanisms 434 can have identical or mirrored configurations.

As shown in FIGS. 4I-4N, the instrument attachment assembly 408 includes a latch member 460 (e.g., a post or sidewall of the instrument) configured to sequentially engage the engagement helix member 414 and the latching helix member 416 to latch to the gear 410 of the carriage attachment assembly 406. The post 460 defines a distal end surface 462 and an indent 464 spaced proximally from the end surface 462 defining a proximal surface 466. FIG. 4L shows the instrument attachment assembly 408, and the latch member 460 thereof, spaced from the carriage attachment assembly 406 with the gears 410 in the open position.

With this configuration, as the post 460 is inserted through the access opening 456, the end surface 462 engages the engagement surface 420 of the engagement helix member 414 to begin rotation of the gear 410 as shown in FIG. 4M. As the post 460 continues to be inserted, the end surface 462 slides down the engagement surface 420, causing the gear 410 to rotate away from the open position towards the latched position. The engagement helix member 414 is sized so that full engagement of the end surface 462 of the post 460 with the engagement surface 420 causes the gear 410 to rotate past the neutral position of the biasing mechanism 424. Thereafter, the biasing mechanism 424 urges the gear 410 to the latched position. Further, when the post 460 reaches the end of the engagement helix member 414, the post 460 is inserted through the gap 418 between the engagement and latching helix members 414, 416 to align the indent 464 with the latching helix member 416. The biasing force of the biasing mechanism 424 causes the latching helix member 416 to rotate into the indent 464 and, as such, causes the proximal surface 466 of the indent 464 to engage the latch surface 422 of the latching helix member 416. As the gear 410 further rotates, engagement of the angled latch surface 422 to the proximal surface 466 tightens the coupling of the carriage attachment assembly 406 to the instrument attachment assembly 408 until the carriage attachment assembly 406 and the instrument attachment assembly 408 are fully seated together in the latched position as shown in FIG. 4N.

In some examples, the end surface 462 and/or proximal surface 466 are angled to be complementary with or extend along the same angle as the engagement surface 420 or latch surface 422, respectively. As such, the end surface 462 can be angled to be equal to or within about 1 degree to about 20 degrees of the ramp angle of the engagement surface 420. The proximal surface 466 can be angled to be equal to or within about 1 degree to about 3 degrees of the ramp angle of the latch surface 422.

The post 460 can be configured to interface with multiple gears 410, if desired. For example, as shown in FIG. 4I, the post 460 has a symmetrical configuration where the end surface 462 is divided into side-by-side end surface portions to engage adjacent gears 410. Further, the post 460 includes opposing indents 464 with laterally spaced proximal surface portions 466 configured to engage the adjacent gears 410.

Although the latch member 460 is described and shown as a post, the distal end surface 462, the indent 464, and the proximal surface 466 can alternatively be defined by a wall (e.g., outer wall) of the instrument 404 for a gear to engage and secure the instrument attachment assembly 408 and the carriage attachment assembly 406 together.

As shown in FIG. 4J, the instrument carriage 402 and the instrument 404 have opposing mounting faces 468, 470 that are drawn together as the attachment assemblies 406, 408 are coupled together. In some examples, the instrument 404 defines a cavity 472 in the mounting face 470 sized to receive a gear protrusion 474 of the instrument carriage housing 444, such that the post 460 can be fully inserted into the carriage attachment assembly 406.

As the mounting faces 468, 470 are interfaced together, one or more components of the instrument carriage 402 can be operably coupled to one or more corresponding components of the instrument 404. In some examples, the instrument carriage 402 and the instrument 404 include plug and port connection(s) and/or and include pin and circuit board interface(s) for one or more sensors or components of the instrument 404. These sensors or components can include, for example, one or more shape sensors (e.g., fiber shape sensor), one or more position sensors (EM sensors), one or more camera lines, one or more light lines, and so forth. In additional or alternative examples, the instrument 404 includes a plurality of drive components (e.g., disks having pull wires wound therearound) with a plurality of steering components and the instrument carriage 402 includes drive mechanisms, such as drive motors, that interface with and drive the drive components of the instrument 404. In some examples, the mounting faces 468, 470 can include kinematic mount features that interact to precisely align the instrument carriage 402 and the instrument 404. The kinematic mount features can have any desired configuration, such as a Kelvin coupling (e.g., one of the mounting faces 468, 470 including three arrayed spherical surfaces and the other of the mounting faces 468, 470 including a concave tetrahedron, a V-groove, and a flat plate for six contact points) or a Maxwell coupling (e.g., one of the mounting faces 468, 470 including three arrayed spherical surfaces and the other of the mounting faces 468, 470 including three V-shaped grooves oriented to a center of the face for six contact points).

In some examples, the instrument 404 includes a housing 476, including a cover 478 and a chassis 480. The chassis 480 carries components that interface with the instrument carriage 402 including, for example, steering components, drive components, a support fixture, sensor connectors, and so forth.

The instrument 404 includes an elongate medical device 482 (FIG. 4J) (e.g., a flexible elongate device or a rigid elongate device, such as a scope, catheter, tool, etc.) that is coupled to the housing 476. For example, a proximal portion of the elongate medical device 482 extends through and terminates within the housing 476.

An alternative configuration suitable for the medical system 400 described above is shown in FIG. 4O utilizing two latch members 460′. Rather than a single post as with the above example latch member 460, the latch members 460′ of this example are spaced from one another to engage gears 410 having a corresponding wider spacing relative to one another. This spacing allows components and/or interfaces to be positioned between the connections between the latch members 460′ and the gears 410, such that when the latch member 460′ is secured to the gears 410, the components and/or interfaces are held securely together between the connections. The latch members 460′ can be posts or sidewalls of the instrument as discussed above. Further, the gears 410 can both be disposed laterally outwardly of the latch members 460′, both be disposed laterally inwardly of the latch members 460′, disposed longitudinally above or below the latch members 460′, or a combination thereof.

As with the above example, the latch members 460′ each include a distal end surface 462 and an indent 464 spaced proximally from the end surface 462 defining a proximal surface 466 to sequentially engage the engagement helix member 414 and the latching helix member 416 to latch to the gear 410 of the carriage attachment assembly 406.

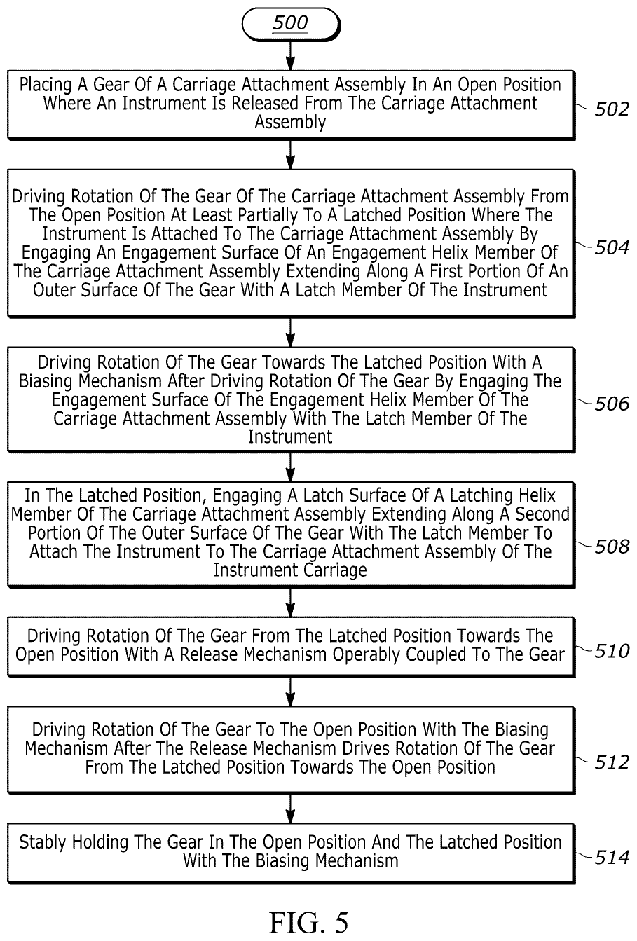

A method 500 for attaching and detaching an instrument (e.g., instrument 404) from a carriage attachment assembly (e.g., carriage attachment assembly 406) of an instrument carriage (e.g., instrument carriage 402) is shown in FIG. 5.

In step 502, the method 500 includes placing a gear (410) of the carriage attachment assembly in an open position. For example, step 502 can include rotating or otherwise disposing the gear in the open position, where an engagement helix member (e.g., engagement helix member 414) is accessible for engagement by the instrument and the instrument is released from the carriage attachment assembly. The method 500 further includes, in step 504, driving rotation of the gear from the open position at least partially to a latched position where the instrument is attached to the carriage attachment assembly by engaging an engagement surface (e.g., engagement surface 420) of the engagement helix member of the carriage attachment assembly extending along a first portion of an outer surface (e.g., outer surface 412) of the gear with a latch member (e.g., post 460) of the instrument. For example, the latch member may be pushed along the gear to slide down the engagement helix member, which causes the gear to rotate away from the open position towards the latched position.

In step 506, the method 500 further includes driving rotation of the gear towards the latched position with a biasing mechanism (e.g., biasing mechanism 424) after driving rotation of the gear by engaging the engagement surface of the engagement helix member of the carriage attachment assembly with the latch member of the instrument.

In step 508, in the latched position, the method 500 includes engaging a latch surface (e.g., latch surface 422) of a latching helix member (e.g., latching helix member 416) of the carriage attachment assembly extending along a second portion of the outer surface of the gear with the latch member to attach the instrument to the carriage attachment assembly of the instrument carriage. In the latched position, the instrument is securely attached to the carriage attachment assembly.

The instrument may be used, such as for a medical procedure, while attached to the instrument carriage. After usage, the instrument may be unattached from the instrument carriage as discussed in steps 510 and 512. In step 510, the method 500 includes driving rotation of the gear from the latched position towards the open position with a release mechanism (e.g., release mechanism 432) operably coupled to the gear and, in step 512, driving rotation of the gear to the open position with the biasing mechanism after the release mechanism drives rotation of the gear from the latched position towards the open position. In step 514, the instrument is moved away from the instrument carriage after the gear is rotated to the open position.

One or more components of the embodiments discussed in this disclosure, such as control system 112, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

1. A medical system, comprising:
an instrument carriage configured to control movement of an instrument, the instrument carriage comprising a carriage attachment assembly configured to removably attach to the instrument, the carriage attachment assembly comprising:
a gear comprising a body having an outer surface;
an engagement helix member of the gear extending along a first portion of the outer surface of the body, the engagement helix member having an engagement surface configured to facilitate rotation of the gear from an open position where the instrument is unattached from the carriage attachment assembly towards a latched position where the instrument is attached to the carriage attachment assembly; and
a latching helix member of the gear extending along a second portion of the outer surface of the body, the latching helix member comprising a latch surface configured to engage the instrument in the latched position.

2. The medical system of claim 1, further comprising a biasing mechanism configured to facilitate rotation of the gear between the open position and the latched position.

3. The medical system of claim 2, wherein the biasing mechanism comprises:
a journal of the gear, the journal having an eccentric configuration relative to a rotational axis of the gear; and
a bias member engaging the journal, the eccentric configuration of the journal causing the bias member to facilitate rotation of the gear towards the latched position in a first range of gear rotation and facilitate rotation of the gear towards the open position in a second range of gear rotation.

4. The medical system of claim 2, wherein the biasing mechanism is stable in the open position and the latched position.

5. The medical system of claim 1, further comprising a motor configured to facilitate rotation of the gear between the latched position and the open position.

6. The medical system of claim 1, wherein at least one of:
the engagement helix member and the latching helix member are circumferentially spaced from one another about the body of the gear;
a ramp angle of the engagement helix member is larger than a ramp angle of the latching helix member;
the proximal surface has a complementary angle to a ramp angle of the engagement helix member; or
the end surface has a complementary angle to a ramp angle of the latching helix member.

7.-8. (canceled)

9. The medical system of claim 1, further comprising a release mechanism operably coupled to the gear and configured to facilitate rotation of the gear from the latched position towards the open position.

10. The medical system of claim 9, further comprising a second journal of the gear having an eccentric configuration relative to a rotational axis of the gear, the release mechanism coupled to the second journal.

11. The medical system of claim 9, further comprising a biasing mechanism configured to facilitate rotation of the gear between the open position and the latched position, wherein the biasing mechanism is configured to at least partially facilitate rotation of the gear to the open position after the release mechanism facilitates rotation of the gear from the latched position towards the open position.

12.-13. (canceled)

14. The medical system of claim 1, wherein the gear comprises a first gear, and further comprising a second gear disposed adjacent to the first gear, the first gear and the second gear having mirrored configurations.

15. The medical system of claim 1, further comprising an instrument attachment assembly coupled to an instrument, the instrument attachment assembly comprising a post including an end surface and an indent spaced proximally from the end surface defining a proximal surface, the post configured to engage the engagement helix member and the latching helix member of the gear to secure the instrument attachment assembly to the carriage attachment assembly.

16.-18. (canceled)

19. A medical system, comprising:
an instrument attachment assembly coupled to an instrument, the instrument attachment assembly comprising a post including an end surface and an indent proximally spaced from the end surface defining a proximal surface; and
an instrument carriage configured to control movement of the instrument, the instrument carriage comprising a carriage attachment assembly configured to removably attach to the instrument via the instrument attachment assembly, the carriage attachment assembly comprising:
a gear comprising a body having an outer surface;
an engagement helix member of the gear extending along a first portion of the outer surface of the body, the engagement helix member having an engagement surface configured to be engaged by the end surface of the post when force is applied to bring the instrument attachment assembly toward the instrument carriage to facilitate rotation of the gear from an open position where the instrument is released from the carriage attachment assembly towards a latched position where the instrument attachment assembly is attached to the carriage attachment assembly; and
a latching helix member of the gear extending along a second portion of the outer surface of the body, the latching helix member comprising a latch surface configured to engage the proximal surface of the post in the latched position.

20. The medical system of claim 19, wherein the carriage attachment assembly further comprises a biasing mechanism configured to facilitate rotation of the gear between the open position and the latched position.

21. The medical system of claim 20, wherein the biasing mechanism comprises:
a journal of the gear, the journal having an eccentric configuration relative to a rotational axis of the gear; and
a bias member engaging the journal, the eccentric configuration of the journal causing the bias member to facilitate rotation of the gear towards the latched position in a first range of gear rotation and facilitate rotation of the gear towards the open position in a second range of gear rotation.

22.-26. (canceled)

27. The medical system of claim 19, wherein the carriage attachment assembly further comprises a release mechanism operably coupled to the gear and configured to facilitate rotation of the gear from the latched position towards the open position.

28. The medical system of claim 27, wherein the carriage attachment assembly further comprises a second journal of the gear having an eccentric configuration relative to a rotational axis of the gear, the release mechanism coupled to the second journal.

29. The medical system of claim 27, wherein the carriage attachment assembly further comprises a biasing mechanism configured to facilitate rotation of the gear between the open position and the latched position, wherein the biasing mechanism is configured to at least partially facilitate rotation of the gear to the open position after the release mechanism facilitates rotation of the gear from the latched position towards the open position.

30.-31. (canceled)

32. The medical system of claim 19, wherein the gear comprises a first gear, and the carriage attachment assembly further comprises a second gear disposed adjacent to the first gear, the first gear and the second gear having mirrored configurations.

33. (canceled)

34. A medical system, comprising:
an instrument comprising:
an elongate medical device; and
an instrument attachment assembly coupled to the elongate medical device and configured to removably attach to an instrument carriage for movement control of the elongate medical device, the instrument attachment assembly comprising a post including an end surface configured to facilitate rotation of a gear of the instrument carriage from an open position where the post is released from the instrument carriage towards a latched position where the post is attached to the instrument carriage and an indent spaced from the end surface and defining a proximal surface, the proximal surface configured to engage the gear in the latched position.

35. The medical system of claim 34, wherein the end surface of the post comprises angled first and second end surface portions, the first end surface portion configured to facilitate rotation of a first gear of the instrument carriage from the open position towards the latched position and the second end surface portion configured to facilitate rotation of a second gear of the instrument carriage from the open position towards the latched position; and the proximal surface of the post includes first and second proximal surface portions, the first proximal surface portion configured to engage a first gear of the instrument carriage in the latched position and the second proximal surface portion configured to engage a second gear of the instrument carriage in the latched position.

36. (canceled)

37. The medical system of claim 34, wherein the post comprises a first post; and further comprising a second post spaced from the first post, the second post including an end surface configured to facilitate rotation of a second gear of the instrument carriage from an open position where the second post is released from the instrument carriage towards a latched position where the second post is attached to the instrument carriage and an indent spaced from the end surface and defining a proximal surface, the proximal surface configured to engage the second gear in the latched position

38. The medical system of claim 34, wherein the instrument further comprises a housing with a chassis configured to carry components that interface with the instrument carriage.

39. The medical system of claim 34, wherein a proximal portion of the elongate medical device extends through and terminates within the housing.

40.-46. (canceled)
